(19) United States
(12) Patent Application Publication
(10) Pub. No.: US 2018/0298292 A1
(43) Pub. Date: Oct. 18, 2018

(54) CYROGENIC SEPARATION OF LIGHT OLEFINS AND METHANE FROM SYNGAS

(71) Applicant: SABIC GLOBAL TECHNOLOGIES B.V., Bergen op Zoom (NL)

(72) Inventors: Thabet Al-Qahtani, Riyadh (SA); Khalid Karim, Riyadh (SA); Mubarik Ali Bashir, Riyadh (SA); Ali Al-Hammad, Riyadh (SA)

(21) Appl. No.: 15/763,683

(22) PCT Filed: Sep. 28, 2016
(86) PCT No.: PCT/IB2016/055807

§ 371 Date: Mar. 27, 2018

Related U.S. Application Data
(60) Provisional application No. 62/234,091, filed on Sep. 29, 2015.
(60) Provisional application No. 62/234,093, filed on Sep. 29, 2015.
(60) Provisional application No. 62/234,096, filed on Sep. 29, 2015.

(51) Int. Cl. C10G 70/04; C01B 3/50; C07C 7/00; C07C 7/04; C07C 7/09; F25J 3/02

(52) U.S. Cl. CPC C10G 70/043; F25J 2270/42; C07C 7/005; C07C 7/04; C07C 7/09; F25J 3/0219; F25J 3/0233; F25J 3/0271; C01B 2203/0233; C01B 2203/0244; C01B 2203/0261; C01B 2203/046; C01B 2203/062; F25J 2200/74; F25J 2200/94; F25J 2205/04; F25J 2210/12; C01B 3/506

(57) ABSTRACT

In accordance with the present invention, disclosed herein is a method comprising the steps for separating syngas and methane from C2-C4 hydrocarbons. Also disclosed herein, are systems utilized to separate syngas and methane from C2-C4 hydrocarbons.

### CROSS-REFERENCE TO RELATED APPLICATIONS

This Application claims the benefit of U.S. Provisional Application Nos. 62/234,091, filed Sep. 29, 2015; 62/234,093, filed Sep. 29, 2015; and 62/234,096, filed Sep. 29, 2015, which are all incorporated herein by reference in their entirety.

### BACKGROUND

Syngas (mixtures of H₂ and CO) can be readily produced from either coal or methane (natural gas) by methods well known in the art and widely commercially practiced around the world. A number of well-known industrial processes use syngas for producing various hydrocarbons and oxygenated organic chemicals.

The Fischer-Tropsch catalytic process for catalytically producing hydrocarbons from syngas was initially discovered and developed in the 1920's, and was used in South Africa for many years to produce gasoline range hydrocarbons as automotive fuels. The catalysts typically comprised iron or cobalt supported on alumina or titania, and promoters, like rhenium, zirconium, manganese, and the like were sometimes used with cobalt catalysts, to improve various aspects of catalytic performance. The products were typically gasoline-range hydrocarbon liquids having six or more carbon atoms, along with heavier hydrocarbon products.

Today lower molecular weight C2-C4 hydrocarbons are desired and can be obtained from syngas via the Fischer-Tropsch catalytic process. Challenges exist to efficiently separate unreacted syngas and methane from the lower molecular weight C2-C4 hydrocarbons. Furthermore, to ensure highest yields it is also desirable to recycle unreacted syngas and the separated methane back to the Fischer-Tropsch catalytic process.

Accordingly, there remains a long-term market need for new and improved methods for separation of light olefins and methane from syngas. Still further, there is a need for recycling the separated syngas back to the Fischer-Tropsch catalytic process, and utilizing the separated methane to further generate additional syngas used in the process.

Accordingly, a system and method useful for the separation of C2-C4 hydrocarbons from a produced from syngas are described herein.

### SUMMARY OF THE INVENTION

Disclosed herein is a method comprising the steps of: a) providing a first product stream comprising syngas, methane, and C2-C4 hydrocarbons, wherein the first product stream has a first temperature; b) lowering the first temperature of the first product stream to a second temperature in a first heat exchange unit; c) separating at least a portion of the syngas from the first product stream in a cryogenic separation unit, thereby producing a second product stream comprising methane and C2-C4 hydrocarbons; d) separating at least a portion of the methane in the second product stream in a demethanizer, thereby producing a third product stream comprising C2-C4 hydrocarbons; e) recycling the at least a portion of the separated syngas to a Fischer-Tropsch reactor; and f) recycling the at least a portion of the separated methane to a syngas generation unit.

Also disclosed herein is a system comprising: a) a syngas generation unit; b) a Fisher-Tropsch reactor; c) a first, a second, a third, and a fourth heat exchange unit, wherein at least one of the second, the third, and the fourth exchange units is in communication with the first heat exchange unit; d) a first refrigeration unit, wherein the first refrigeration unit is in communication with the first heat exchange unit; e) a cryogenic separation unit, wherein the cryogenic separation unit is in communication with the Fisher-Tropsch reactor; and f) a demethanizer, wherein the demethanizer is in communication with the syngas generation unit.

Additional advantages will be set forth in part in the description which follows, and in part will be obvious from the description, or can be learned by practice of the aspects described below. The advantages described below will be realized and attained by means of the chemical compositions, methods, and combinations thereof particularly pointed out in the appended claims. It is to be understood that both the foregoing general description and the following detailed description are exemplary and explanatory only and are not restrictive of the invention, as claimed.

### DESCRIPTION OF THE FIGURES

The accompanying figures, which are incorporated in and constitute a part of this specification, illustrate several aspects, and together with the description, serve to explain the principles of the invention.

FIG. 1 shows a flow diagram of a system and a method described herein.

FIG. 2 shows a flow diagram of a system and a method described herein.

FIG. 3 shows a flow diagram of a system and a method described herein.

The present invention can be understood more readily by reference to the following detailed description of the invention.

### DETAILED DESCRIPTION

The present invention can be understood more readily by reference to the following detailed description of the invention.

Disclosed herein are materials, compounds, compositions, and components that can be used for, can be used in conjunction with, can be used in preparation for, or are products of the disclosed method and compositions. It is to be understood that when combinations, subsets, interactions, groups, etc. of these materials are disclosed that while specific reference of each various individual and collective combinations and permutation of these compounds cannot be explicitly disclosed, each is specifically contemplated and described herein. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

All publications mentioned herein are incorporated herein by reference to disclose and describe the methods and/or materials in connection with which the publications are cited.

### 1. Definitions

In this specification and in the claims which follow, reference will be made to a number of terms which shall be defined to have the following meanings:

As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “a hydrocarbon” includes mixtures of two or more hydrocarbons.

As used herein, the terms “about” and “at or about” mean that the amount or value in question can be the value designated some other value approximately or about the same. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The terms “first,” “first product stream,” “first heat exchange unit,” “second,” “second product stream,” “second heat exchange unit,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not.

References in the specification and concluding claims to parts by weight, of a particular element or component in a composition or article, denote the weight relationship between the element or component and any other elements or components in the composition or article for which a part by weight is expressed. Thus, in a compound containing 2 parts by weight of component X and 5 parts by weight of component Y, X and Y are present at a weight ratio of 2:5, and are present in such a ratio regardless of whether additional components are contained in the compound.

A weight percent (“wt %”) of a component, unless specifically stated to the contrary, is based on the total weight of the formulation or composition in which the component is included. For example, if a particular element or component in a composition or article is said to have about 80% by weight, it is understood that this percentage is relative to a total compositional percentage of 100% by weight.

A mole percent (“mole %”) of a component, unless specifically stated to the contrary, is based on the total number of moles of all chemical components present in the formulation or composition in which the component is included. For example, if a particular element or component in a composition is said to be present in amount about 1 mole %, it is understood that this percentage is relative to a total compositional percentage of 100% by mole.

As used herein, the terms “syngas” or “synthesis gas” are used interchangeably herein.

It is to be understood that the term “at least a portion,” can be “at least a first portion,” “at least a second portion,” “at least a third portion,” “at least a fourth portion,” or “at least a fifth portion,” or the like. For example, the reference of an “at least a first portion” can be used to distinguish it from an “at least a second portion.” Such as, for example, the reference of an “at least a first portion of the separated syngas” can be used to distinguish it from an “at least a second portion of the separated syngas.”

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

### 2. System

Disclosed herein is a system comprising: a) a syngas generation unit; b) a Fisher-Tropsch reactor; c) a first, a second, a third, and a fourth heat exchange unit, wherein at least one of the second, the third, and the fourth exchange units is in communication with the first heat exchange unit; d) a first refrigeration unit, wherein the first refrigeration unit is in communication with the first heat exchange unit; e) a cryogenic separation unit, wherein the cryogenic separation unit is in communication with the Fisher-Tropsch reactor; and f) a demethanizer, wherein the demethanizer is in communication with the syngas generation unit.

In one aspect, the system further comprises a catalytic conversion unit that is in communication with a syngas generation unit. A catalytic conversion unit is known in the art and can upgrade hydrocarbons, such as paraffins, to olefins. For example, the catalytic conversion unit can upgrade hydrocarbons present in the first product stream to olefins.

The syngas generation unit is any unit known in the art capable of generating a synthesis gas (syngas). According to the aspects of this disclosure, the syngas generation unit is in communication with the Fisher-Tropsch reactor and with the methane recovery unit. The Fisher-Tropsch reactor is also in communication with the syngas recovery unit. Isothermal and/or adiabatic fixed bed reactors can be used as a Fischer-Tropsch reactor, which can carry out the Fischer-Tropsch process. The Fischer-Tropsch reactor can comprise one or more Fischer-Tropsch catalysts. Fischer-Tropsch catalysts are known in the art and can, for example, be Fe based catalysts and/or Co based catalysts and/or Ru based catalysts.

In one aspect, the first heat exchange unit is in communication with the second heat exchange unit. In another aspect, the first heat exchange unit is in communication with the third heat exchange unit. In yet another aspect, the first heat exchange unit is in communication with the fourth heat exchange unit. In a further aspect, the first heat exchange unit is in communication with the second, the third and the fourth heat exchange units.

In one aspect, the cryogenic separation unit comprises at least one distillation column. The cryogenic separation unit is used to separate unreacted syngas from methane and other light hydrocarbons. According to the aspects of this disclosure, the demethanizer can be utilized to separate methane from the C2+ hydrocarbons, such as C2-C4 or C2-C7 hydrocarbons. It is further understood that the cryogenic unit can be in communication with the demethanizer.

In some aspects, the Fisher-Tropsch reactor described herein is in communication with the demethanizer. In other aspects, the communication between the Fisher-Tropsch reactor and the demethanizer is by means of the first heat exchange unit. According to the aspects of the present disclosure, any methane wash units known in the art can be utilized. The methane wash unit is configured to separate hydrogen from a mixture of hydrogen and carbon monoxide, which can be present in the syngas and from methane. In some aspects, the methane wash unit is in communication with the cryogenic separation unit. In other aspects, the methane wash unit is in communication with the demethanizer. In further aspects, the N₂ refrigeration loop comprises a nitrogen refrigeration unit, wherein the nitrogen is a liquid nitrogen. The N₂ refrigeration loop can further comprise pipes, tanks, pumps, valves and any other articles known in the art allowing a flow of nitrogen through the loop.

Optionally, in various aspects, the disclosed system can be operated or configured on an industrial scale. In one aspect, the reactors described herein can each be an industrial size reactor. For example, the syngas generation unit can be an industrial size reactor. In yet another example, the Fischer-Tropsch reactor can be an industrial size reactor. For example, the cryogenic separation unit can be an industrial size reactor. In yet another example, the demethanizer can be an industrial size reactor. In yet further examples, the N₂ refrigeration loop can be an industrial size reactor, and, optionally, the methane wash unit can be an industrial size reactor.

The reactors, units, and vessels disclosed herein can have a volume of at least about 1,000 liters, about 2,000 liters, about 5,000 liters, or about 20,000 liters. For example, the reactor can have a volume from about 1,000 liters to about 20,000 liters.

In one aspect, the syngas generation unit can have a volume of at least about 1,000 liters, about 2,000 liters, about 5,000 liters, or about 20,000 liters. For example, the syngas generation unit can have a volume from about 1,000 liters to about 20,000 liters.

In one aspect, the Fischer-Tropsch reactor can have a volume of at least about 1,000 liters, about 2,000 liters, about 5,000 liters, or about 20,000 liters. For example, the Fischer-Tropsch reactor can have a volume from about 1,000 liters to about 20,000 liters.

In one aspect, the cryogenic separation unit can have a volume of at least about 1,000 liters, about 2,000 liters, about 5,000 liters, or about 20,000 liters. For example, cryogenic separation unit can have a volume from about 1,000 liter to about 20,000 liters.

In one aspect, the demethanizer can have a volume of at least about 1,000 liters, about 2,000 liters, about 5,000 liters, or about 20,000 liters. For example, the demethanizer can have a volume from about 1,000 liters to about 20,000 liters.

In one aspect, the methane wash unit can have a volume of at least about 1,000 liters, about 2,000 liters, about 5,000 liters, or about 20,000 liters. For example, the methane wash unit can have a volume from about 1,000 liters to about 20,000 liters.

In one aspect, the N₂ refrigeration loop can have a volume of at least about 1,000 liters, about 2,000 liters, about 5,000 liters, or about 20,000 liters. For example, the N₂ refrigeration loop can have a volume from about 1,000 liters to about 20,000 liters.

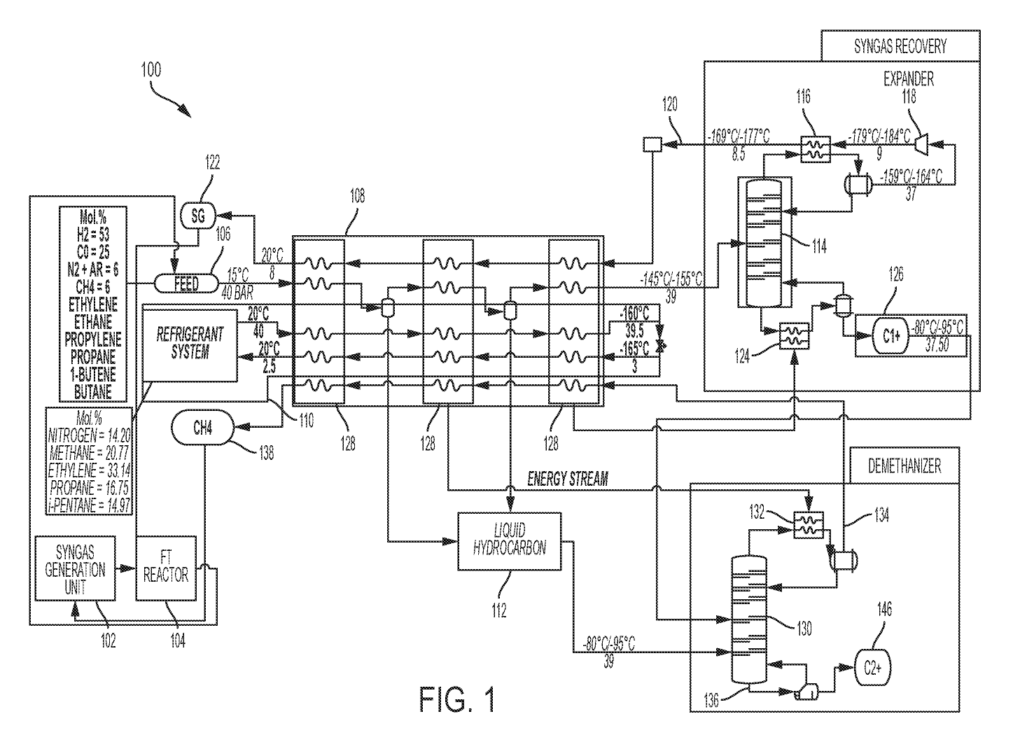

Now referring to FIG. 1, which shows a non-limiting exemplary aspect of the system and method disclosed herein. FIG. 1 shows a system (100). The system has a syngas generation unit (102). The syngas generation unit is in fluid communication with a Fisher-Tropsch reactor (104). The feed (106) comprising a first product stream and exiting Fisher-Tropsch reactor (104) is passing through a first heat exchange unit (108). The first heat exchange unit (108) can comprise one or more heat exchange devices (128). The first product stream passing through the first exchange unit (108) is cooled down and a hydrocarbon product comprising C2-C7 in a liquid form (112) is separated from the first product stream. The temperature of the first product stream is lowered in the first heat exchange unit (108). The liquid hydrocarbon comprising C2-C7 is separated as the first product stream passes through one or more heat exchange devices (128). The first heat exchange unit (108) is in communication with a first refrigeration unit (110). In some aspects, the first heat exchange unit (108) is in thermal communication with the first refrigeration unit (110). The first product stream is separated in a cryogenic separation unit (114), wherein the syngas is separated and a second product stream comprising methane and C2-C4 hydrocarbons is formed. The at least a portion of the separated syngas passes through a second heat exchange unit (116) that is in communication with the first heat exchange unit (108). In some aspects, the second heat exchange unit (116) is in thermal communication with the first heat exchange unit (108). The at least a portion of the separated syngas comprises at least a first portion and at least a second portion. In some aspect, the at least a first portion of the separated syngas exiting the cryogenic separation unit (114) passes through a gas expansion unit (118) that is also in communication with the second heat exchange unit (116). In another aspect, the at least a second portion of the separated syngas is recycled back to the cryogenic separation unit (114). The at least a first portion of the separated syngas is collected in a syngas recovery unit (122) that is in communication with Fisher-Tropsch reactor (104). The second product stream passes through a third heat exchange unit (124) that is in communication with the first heat exchange unit (108). The second product stream comprises at least a first portion and at least a second portion of the second product stream. In some aspects the at least a first portion of the second product stream enters into demethanizer (130) to separate methane from the second product thereby producing a third product stream comprising C2-C4 hydrocarbons. In other aspects, the at least a second portion of the second product stream is recycled back to the cryogenic separation unit (114). In some aspects, the third heat exchange unit (124) is in thermal communication with the first heat exchange unit (108). At least a portion of the separated methane passes through a fourth exchange heat unit (132) that is in communication with the first heat exchange unit (108). At least a portion of the separated methane comprises at least a first portion and at least a second portion. The at least a first portion of the separated methane is recycled to the methane recovery unit (138) that is in communication with the syngas generation unit (102). The at least a second portion of the separated methane is recycled back to the top of the demethanizer (130). The third product stream (136) comprises at least a first portion and at least a second portion. The least a second portion of the third product stream is recycled back to the demethanizer (130). The at least first portion of the third product stream is removed (136, 146) to recover C2-C7 hydrocarbons.

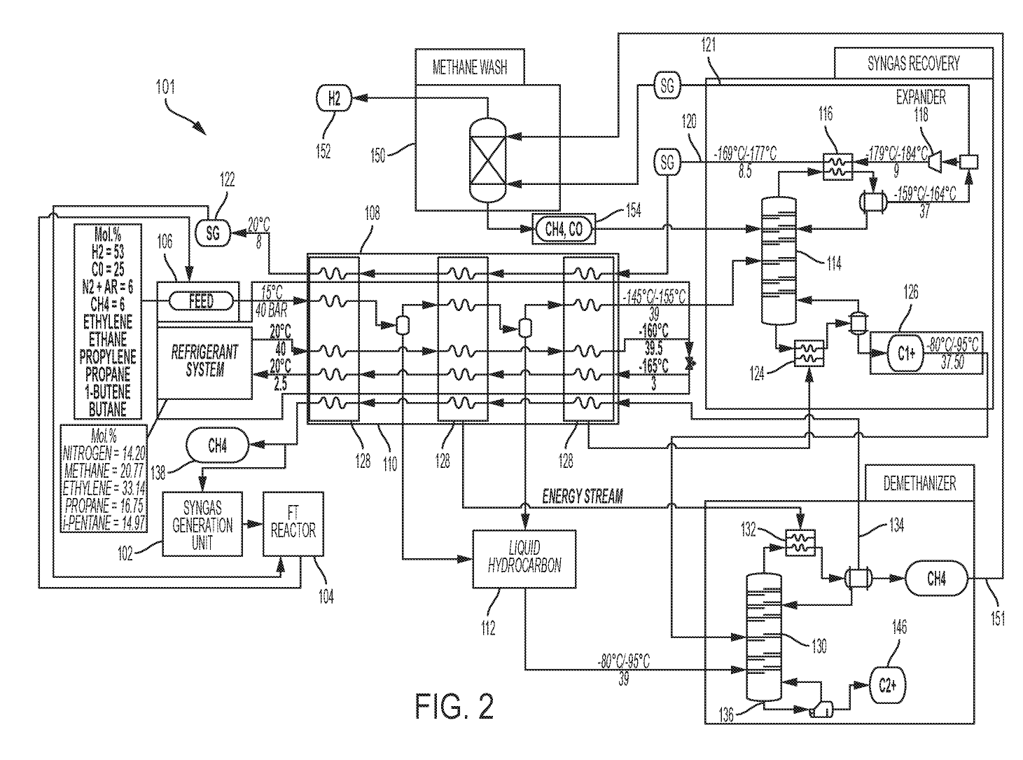

Now referring to FIG. 2, which shows a non-limiting exemplary aspect of the system and method disclosed herein. FIG. 2 shows a system (101). The system has a syngas generation unit (102). The syngas generation unit is in fluid communication with a Fisher-Tropsch reactor (104). The feed (106) comprising a first product stream, comprising syngas, methane and C2-C4 hydrocarbons, and exiting Fisher-Tropsch reactor (104) is passing through a first heat exchange unit (108). The first heat exchange unit (108) can comprise one or more heat exchange devices (128). The first product stream passing through the first exchange unit (108) is cooled down and a hydrocarbon product comprising C2-C7 in a liquid form (112) is separated from the first product stream. A temperature of the first product stream is lowered in the first heat exchange unit (108). The liquid hydrocarbon comprising C2-C7 is separated as the first product stream passes through one or more heat exchange devices (128). The first heat exchange unit (108) is in communication with a first refrigeration unit (110). In some aspects, the first heat exchange unit (108) is in thermal communication with the first refrigeration unit (110). The first product stream is separated in a cryogenic separation unit (114), wherein the syngas is separated from the first product stream and a second product stream comprising methane and C2-C4 hydrocarbons is formed. At least a portion of the separated syngas comprises at least a first portion of separated syngas, at least a second portion of separated syngas, and at least a third portion of separated syngas. The at least a portion of the separated syngas passes through a second heat exchange unit (116) that is in communication with the first heat exchange unit (108). In some aspects, the second heat exchange unit (116) is in thermal communication with the first heat exchange unit (108). In some aspect, the at least a first portion of the separated syngas exiting the cryogenic separation unit (114) passes through a gas expansion unit (118) that is also in communication with the second heat exchange unit (116). In some aspects, the at least a third portion of the separated syngas is recycled back to the cryogenic separation unit (114). In other aspects, the at least a second portion of the separated syngas passes by line (121) to the methane wash unit (150). A methane and carbon monoxide recovery unit (154) is in communication with the methane wash unit (150) and the cryogenic separation unit (114). The methane wash unit (150) is also in communication with a hydrogen recovery unit (152). The at least first portion of the syngas exiting gas expansion unit (118) passes by line (120) through the first heat exchange unit (108) to a syngas recovery unit (122) where it is collected. The syngas recovery unit (122) is in communication with Fisher-Tropsch reactor (104). The second product stream passes through a third heat exchange unit (124) that is in communication with the first heat exchange unit (108). In some aspects, at least a first portion of the second product stream is recycled back to the cryogenic separation unit (114). In other aspects, at least a second portion of the second product stream (126) enters into demethanizer (130) to separate methane from the second product stream thereby producing a third product stream comprising C2-C4 hydrocarbons (136), wherein the third product stream comprising C2-C4 hydrocarbons further comprises at least a first portion and at least a second portion. In some aspects, the third heat exchange unit (124) is in thermal communication with the first heat exchange unit (108). At least a portion of the separated methane comprises at least a first portion, at least a second portion and at least a third portion of the separated methane. The at least a portion of the separated methane passes through a fourth exchange heat unit (132) that is in communication with the first heat exchange unit (108). The at least a first portion of the separated methane is recycled by line (134) to the methane recovery unit (138) that is in communication with the syngas generation unit (102). The at least a second portion of the of the separated methane is transferred to the methane wash unit (150) that is in communication with the cryogenic separation unit (114), methane and carbon monoxide recovery unit (154) and hydrogen recovery unit (152). The at least a third portion of the separated methane is recycled back to the demethanizer (130). At least a first portion of the third product stream is recycled back to the demethanizer (130). At least a second portion of the third product stream is removed (136, 146) to recover C2-C4 hydrocarbons.

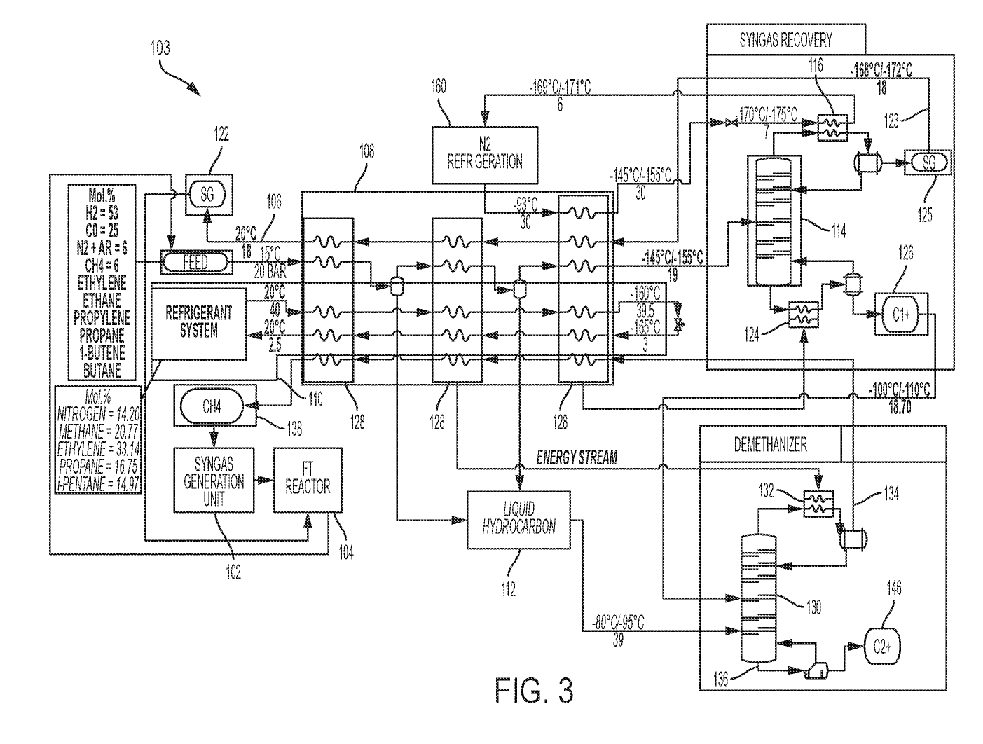

Now referring to FIG. 3, which shows a non-limiting exemplary aspect of the system and method disclosed herein. FIG. 3 shows a system (103). The system has a syngas generation unit (102). The syngas generation unit is in fluid communication with a Fisher-Tropsch reactor (104). The feed (106) comprising a first product stream comprising syngas, methane, and C2-C4 hydrocarbons, and exiting Fisher-Tropsch reactor (104) is passing through a first heat exchange unit (108). The first heat exchange unit (108) can comprise one or more heat exchange devices (128). The first product stream passing through the first exchange unit (108) is cooled down and a hydrocarbon product comprising C2-C7 in a liquid form (112) is separated from the first product stream. A temperature of the first product stream is lowered in the first heat exchange unit (108). The liquid hydrocarbon comprising C2-C7 is separated as the first product stream passes through one or more heat exchange devices (128). The first heat exchange unit (108) is in communication with a first refrigeration unit (110). In some aspects, the first heat exchange unit (108) is in thermal communication with the first refrigeration unit (110). The first product stream is separated in a cryogenic separation unit (114), wherein a syngas is separated from the first product stream and a second product stream comprising methane and C2-C4 hydrocarbons is formed. At least a portion of the separated syngas comprises at least a first portion of the separated syngas and at least a second portion of the separated syngas. At least a portion of the separated syngas comprises at least a first portion and at least a second portion of separated syngas. The at least a first portion of the separated syngas passes through a second heat exchange unit (116) that is in communication with the first heat exchange unit (108). In some aspects, the second heat exchange unit (116) is in thermal communication with the first heat exchange unit (108). The second heat exchange unit (116) is in communication with the nitrogen refrigeration loop (160). In some aspects, the second heat exchange unit (116) is in a thermal communication with a nitrogen refrigeration loop (160). In some aspect, the at least a first portion (125) of the separated syngas exiting the cryogenic separation unit (114) is recycled back by means of line (123) through the first heat exchange unit (108) to a syngas recovery unit (122) where it is collected. The syngas recovery unit (122) is in communication with the Fisher-Tropsch reactor (104). In some aspects, the at least a second portion of the separated syngas is recycled back to the cryogenic separation unit (114). The second product stream passes through a third heat exchange unit (124) that is in communication with the first heat exchange unit (108). In some aspects, the at least a first portion of the second product stream is recycled back to the cryogenic separation unit (114). In other aspects, the at least a second portion of the second product stream (126) enters into the demethanizer (130) to separate methane from the second product stream thereby producing a third product stream comprising C2-C4 hydrocarbons (136). In some aspects, the third heat exchange unit (124) is in thermal communication with the first heat exchange unit (108). At least a portion of the separated methane comprises at least a first portion and at least a second portion of the separated methane. The at least a portion of the separated methane passes through a fourth exchange heat unit (132) that is in communication with the first heat exchange unit (108). The at least a first portion of the separated methane is recycled by line (134) to the methane recovery unit (138) that is in communication with the syngas generation unit (102). The at least a second portion of the separated methane is recycled back to the demethanizer (130). At least a first portion of the third product stream is recycled back to the demethanizer (130). At least a second portion of the third product stream is removed (136, 146) to recover C2-C4 hydrocarbons.

### 3. Methods

Disclosed herein is a method comprising the steps of: a) providing a first product stream comprising syngas, methane, and C2-C4 hydrocarbons, wherein the first product stream has a first temperature; b) lowering the first temperature of the first product stream to a second temperature in a first heat exchange unit; c) separating at least a portion of the syngas from the first product stream in a cryogenic separation unit, thereby producing a second product stream comprising methane and C2-C4 hydrocarbons; d) separating at least a portion of the methane in the second product stream in a demethanizer, thereby producing a third product stream comprising C2-C4 hydrocarbons; e) recycling the at least a portion of the separated syngas to a Fischer-Tropsch reactor; and f) recycling the at least a portion of the separated methane to a syngas generation unit.

In the exemplary aspect, the method disclosed herein is schematically illustrated in FIGS. 1, 2 and 3. In one aspect, the syngas is generated in a syngas generation unit 102. It is understood that the syngas can be generated from a variety of different materials that contain carbon. In some aspects, the syngas can be generated from biomass, plastics, coal, municipal waste, natural gas, or any combination thereof. In yet other aspects, the syngas can be generated from a fuel comprising methane. In some other aspects, the syngas generation from the fuel comprising methane can be based on steam reforming, autothermal reforming, or a partial oxidation, or any combination thereof. In some aspects, the syngas is generated by a steam reforming. In these aspects, steam methane reforming uses an external source of hot gas to heat tubes in which a catalytic reaction takes place that converts steam and methane into a gas comprising hydrogen and carbon monoxide. In other aspects, the syngas is generated by autothermal reforming. In these aspects, methane is partially oxidized in a presence of oxygen and carbon dioxide or steam. In aspects, where oxygen and carbon dioxide are used to generate syngas from methane, the hydrogen and carbon monoxide can be produced in a ratio of 1 to 1. In some aspects, where oxygen and steam are utilized, the hydrogen and carbon monoxide can be produced in a ratio of 2.5 to 1. In some other aspects, the syngas is generated by a partial oxidation. In these other aspects, a substoichiometric fuel-air mixture is partially combusted in a reformer, creating a hydrogen-rich syngas. In certain aspect, the partial oxidation can comprise a thermal partial oxidation and catalytic partial oxidation. In some aspects, the thermal partial oxidation is dependent on the air-fuel ration and proceed at temperatures of 1,200° C. or higher. In yet other aspects, the catalytic partial oxidation use of a catalyst allows reduction of the required temperature to about 800° C. to 900° C. It is further understood that the choice of reforming technique can depend on the sulfur content of the fuel being used. The catalytic partial oxidation can be employed if the sulfur content is below 50 ppm. A higher sulfur content can poison the catalyst, and thus, other reforming techniques can be utilized.

In certain aspects, the syngas generated in the syngas generation unit (102) enters a Fischer-Tropsch reactor (104) wherein the desired hydrocarbons are catalytically produced. It is understood that the first product stream described in the aspects of this disclosure is formed in the Fisher-Tropsch reactor (104). The Fischer-Tropsch catalytic process for producing hydrocarbons from syngas is known in the art. Several reactions can take place in a Fischer-Tropsch process, such as, a Fischer-Tropsch (FT) reaction, a water gas shift reaction, and a hydrogen methanation, as shown in Scheme 1.

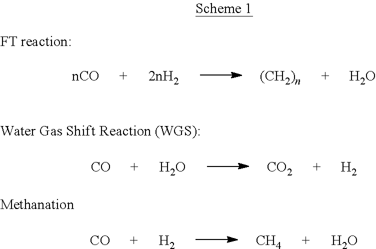

It is understood that the composition of syngas entering a Fischer-Tropsch reactor can vary significantly depending on the feedstock and the gasification process involved. In some aspects, the syngas composition can comprise from about 25 to about 60 wt. % carbon monoxide (CO), about 15 to about 50 wt. % hydrogen (H₂), from 0 to about 25 wt. % methane (CH₄), and from about 5 to about 45 wt. % carbon dioxide (CO₂). In yet other aspects, the syngas can further comprise nitrogen gas, water vapor, sulfur compounds such as for example, hydrogen sulfide (H₂S) and carbonyl sulfide (COS). In yet other aspects, the syngas can further comprise ammonia and other trace contaminants.

The main gases that are being mixed in the Fischer-Tropsch process described herein comprise H₂ and CO. In some aspects, the H₂/CO molar ratio of the feed can be from about 0.5 to about 4. In some exemplary aspects, the H₂/CO molar ratio can be from about 1.0 to about 3.0. In other exemplary aspects, the H₂/CO molar ratio can be from about 1.5 to about 3.0, or yet further exemplary aspects, the H₂/CO molar ratio can be from about 1.5 to about 2.5. It will be appreciated that the H₂/CO molar ratio can control the selectivity of the hydrocarbons that are being produced. The consumption molar ratio of H₂/CO is usually from about 1.0 to about 2.5, such as for example, from about 1.5 to 2.1. This ratio increases as long as the water gas shift reaction is active, and thus, the use of a feed ratio below the consumption ratio will result in a stable H₂/CO ratio during the reaction within an acceptable range (normally below about 2). The H₂ and CO are catalytically reacted in a Fischer-Tropsch reaction.

A Fischer-Tropsch process that targets the production of light olefins (C2-C8 olefins) is desired and such process can produce a significant amount of C2-C4 hydrocarbons. In some aspects, the feed exiting from the Fischer-Tropsch reactor comprises a first product stream (106). In some aspects, the first product stream comprises syngas, methane and C2-C4 hydrocarbons. In some exemplary aspects, the first product stream can comprise hydrogen, carbon monoxide, methane, ethylene, ethane, propylene, propane, butene, butane, mixture of nitrogen and argon, C2-C7 hydrocarbons or any combination thereof. It is understood that all components present in the first product stream can be in any ratio relatively to each other. This first product stream is further processed to separate methane and C2-C4 hydrocarbons from the unreacted syngas. An exemplary non-limiting composition of the first product steam is shown in Table 1. The values shown in Table 1 were simulated using Aspen HYSYS V8.4. The values in Table 1 of the first product stream were calculated after removal of CO₂ and upgrade of C4-C9 hydrocarbons (olefins) via a catalytic conversion unit before being integrated with the remainder of the system disclosed herein.

TABLE 1

| First Product Stream Components | Wt. % |
|---|---|
| CO | 40-55 |
| H₂ | 8-12 |
| Methane | 7-11 |
| C2-C7 Olefins/paraffin's | 21-30 |

In some aspects, the first product stream has a first temperature. In certain aspect, the first temperature is in the range from about 10° C. to about 50° C., including exemplary values of about 15° C., about 20° C., about 25° C., about 30° C., about 35° C., about 40° C., and about 45° C. Still further, the first temperature can be in any range derived from any two of the above stated values. For example, the first product stream can have a first temperature in the range from about 15° C. to about 40° C., or from about 20° C. to about 50° C.

In certain aspects, the first product stream can have a first pressure. In one aspect, the first pressure is in the range from about 1 bar to about 70 bar, including exemplary values of about 5 bar, about 10 bar, about 15 bar, about 20 bar, about 25 bar, about 30 bar, about 35 bar, about 40 bar, about 50 bar, about 55 bar, about 60 bar, and about 65 bar. Still further, the first pressure can be in any range derived from any two of the above stated values. For example, the first product stream can have a first pressure in the range from about 20 bar to about 50 bar, or from about 40 bar to about 65 bar.

In further aspects, the first temperature of the first product stream can be lowered to a second temperature in a first heat exchange unit (108). In some aspects, the second temperature is in the range from about −120° C. to about −170° C., including exemplary values of about −125° C., about −130° C., about −135° C., about −140° C., about −145° C., about −150° C., about −155° C., about −160° C., and about −165° C. Still further, the second temperature can be in any range derived from any two of the above stated values. For example, the second temperature can be in the range from about −130° C. to about −155° C., or from about −145° C. to about −160° C.

The heat exchange units are known in the art. In some aspects, the first heat exchange unit can comprise one or more heat exchange devices (128). In other aspects, the first heat exchange unit can comprise at least two heat exchange devices. It is further understood that the term “heat exchange device,” as used herein, refers to any device built for efficient heat transfer from one medium to another. In some aspects, the media can be separated by a solid wall to prevent mixing. In other aspects, the media can be in direct contact. It is understood that any known in the art heat exchange devices can be used in the method disclosed herein.

It is further understood that the heat exchange devices can be classified according to their flow arrangements. In the aspects, where two fluids enter the exchanger at the same end, and travel in parallel to one another to the other side, the heat exchange device is classified as parallel-flow heat exchanger. In the aspects, where two fluids enter the exchanger from opposite ends is classified as a counter-flow heat exchanger. In the aspects, where two fluids travel perpendicular to one another through the exchange, the heat exchange device is classified as a cross-flow heat exchanger. It is understood that the first heat exchange unit can comprise one or more of a parallel-flow heat exchange device, a counter-flow heat exchange device, or a cross-flow heat exchange device, or any combinations thereof. In some aspects, the first heat exchange unit is one or more of a parallel-flow heat exchange device. In another aspect, the first heat exchange unit is one or more of a counter-flow heat exchange device. In a yet further aspect, the first heat exchange unit is one or more of a cross-flow heat exchange device.

In some aspects, to accelerate the heat exchange process, the first heat exchange unit can be in communication with a first refrigeration system (110). The first refrigeration system can comprise a variety of gases. In some aspects, the first refrigeration system can comprise nitrogen, methane, ethylene, propane, pentane or any combinations thereof.

In some aspects, the lowering of the first temperature of the first product stream to the second temperature can separate a hydrocarbon product in a liquid form (112) from the first product stream. In some aspects, the lowering of the first temperature of the first product stream is a staggering process. For example and without limitations, a first lowering of the first temperature of the first product stream occurs in a first heat exchange device; a second lowering of the first temperature of the first product stream occurs in a second heat exchange device, and so forth. It is further understood that in certain aspects, the lowering of the first temperature of the first product stream results in the formation of a hydrocarbon product in a liquid form. In certain aspect, the hydrocarbon product separation can occur when the first product stream passes through each of the heat exchange devices. In certain aspects, the liquid hydrocarbon product can comprise, for example and without limitation, straight chain C2-C7 hydrocarbons. In other aspects, the liquid hydrocarbon product can comprise methane, C2-C7, or any combination thereof. In some other aspects, at least a portion of the liquid hydrocarbon product is further fed to a demethanizer reactor (130) to separate methane from C2-C7 hydrocarbons.

In certain aspects, the first product stream without the separated liquid hydrocarbon products existing the first heat exchange unit at the second temperature enters a cryogenic separation unit (114) to separate at least a portion of the syngas from the first product stream, thereby producing a second product stream comprising methane and C2-C4 hydrocarbons. Optionally, the at least a portion of the separated syngas comprises at least a first, at least a second and at least a third portion of separated syngas. Cryogenic separation units are known in the art. In some aspects, the cryogenic separation comprises at least one distillation column. A non-limiting example of a cryogenic separation unit is described in published US application 2009/0205367 to Price, which is incorporated in its entirety by reference herein, particularly for its disclosure related to cryogenic separation units.

The at least a portion of the separated syngas passes through a second heat exchange unit (116) to arrive at a temperature in the range from about −150° C. to about −170° C., including exemplary values of about −151° C., about −152° C., about −153° C., about −154° C., about −155° C., about −156° C., about −157° C., about −158° C., about −159° C., about −160° C., about −161° C., about −162° C., about −163° C., about −164° C., about −165° C., about −166° C., about −167° C., about −168° C., and about −169° C. Still further, the temperature can be in any range derived from any two of the above stated values. For example, the at least a portion of the separated syngas exiting the second heat exchange unit can have a temperature in the range from about −152° C. to about −169° C., or from about −159° C. to about −164° C.

In certain aspects, the at least a first portion of the separated syngas can be further cooled by lowering a temperature of the at least a portion of the separated syngas to a temperature in the range of about −170° C. to about −200° C., including exemplary values of about −171° C., about −172° C., about −173° C., about −174° C., about −175° C., about −176° C., about −177° C., about −178° C., about −179° C., about −180° C., about −181° C., about −182° C., about −183° C., about −184° C., about −185° C., about −186° C., about −187° C., about −188° C., about −189° C., about −190° C., about −191° C., about −192° C., about −193° C., about −194° C., about −195° C., about −196° C., about −197° C., about −198° C., and about −199° C. Still further, the temperature can be in any range derived from any two of the above stated values. For example, the lowering of the at least a first portion of the separated syngas is to a temperature in the range from about −175° C. to about −195° C., or from about −179° C. to about −184° C.

In certain aspects the lowering of the temperature of the at least a first portion of the separated syngas is performed in a gas expansion unit (118). Gas expansion units are known in the art. A gas expansion unit can, for example, be a turboexpander. A gas expansion unit expands high pressured gas to produce work. Because work is extracted from the expanding high pressure gas, the expansion is approximated by an isentropic process (i.e., a constant entropy process) and the lower pressure exhaust gas from the gas expansion unit is at a low temperature, such as, for example, a temperature from about −170° C. to about −200° C. In some other aspects, the at least a first portion of the separated syngas exiting the gas expansion unit can further pass through the second heat exchange unit (116). In some aspects, the passing of the at least a first portion of the separated syngas exiting the gas expansion unit through the second heat exchange unit transfers its energy to the at least a third portion of the separated syngas that can be returned back to the cryogenic separation unit (114). It is further understood that the second heat exchange unit can comprise one or more of a parallel-flow heat exchange device, a counter-flow heat exchange device, a cross-flow heat exchange device, or any combination thereof.

In yet other aspect, the at least a third portion of the separated syngas can be returned back to the cryogenic separation unit (114) to provide auxiliary reflux.

In some aspects, at least a portion of the separated syngas exiting the cryogenic separation unit has a temperature from about 165° C. to about 175° C., including exemplary values of 169° C. The at least a portion of the separated syngas passes through a second heat exchange unit at (116) that is in thermal communication with nitrogen (N₂) refrigeration loop (160) Nitrogen refrigeration loop (160) provides a flow of a liquid nitrogen having a temperature in the range from about 169° C. to about −200° C., including exemplary values of about −170° C., about −175° C., about −180° C., about −185° C., about −190° C. and about −195° C. Still further, the temperature can be in any range derived from any two of the above stated values. For example, the flow of liquid nitrogen in the nitrogen refrigeration loop can be from about −165° C. to about −180° C., or from about −170° C. to about −175° C.

In some aspects, the flow of a liquid nitrogen from the nitrogen refrigeration loop (160) enters the second heat exchange unit in a parallel flow with the at least a portion of the separated syngas. In some aspects, the flow of a liquid nitrogen enters the second heat exchange unit in a counter-flow with the at least a portion of the separated syngas. It is further understood that the third temperature of the at least a portion of the separated syngas entering the second heat exchange unit (116) is lowered by the heat exchange with the nitrogen refrigeration loop (160); thereby lowering the third temperature to a fourth temperature. It is further understood that the second heat exchange unit can comprise one or more of a parallel-flow heat exchange device, a counter-flow heat exchange device, a cross-flow heat exchange device, or any combination thereof.

The nitrogen refrigeration loops are known in the art. The nitrogen refrigeration loop can comprise pipes, tanks, valves, pumps, or any other known in the art articles and means that allow flow of a liquid nitrogen through the loop to further lower the temperature of a desired gas flow. A non-limiting example of a nitrogen refrigeration loop unit is described in U.S. Pat. No. 6,298,688 to Brostow, which is incorporated in its entirety by reference herein, particularly for its disclosure related to nitrogen refrigeration loops.

In some aspects, the fourth temperature is the range of −150° C. to about −200° C., including exemplary values of about −151° C., about −152° C., about −153° C., about −154° C., about −155° C., about −156° C., about −157° C., about −158° C., about −159° C., about −160° C., about −161° C., about −162° C., about −163° C., about −164° C., about −165° C., about −166° C., about −167° C., about −168° C., about −169° C., about −170° C., about −171° C., about −172° C., about −173° C., about −174° C., about −175° C., about −176° C., about −177° C., about −178° C., about −179° C., about −180° C., about −181° C., about −182° C., about −183° C., about −184° C., about −185° C., about −186° C., about −187° C., about −188° C., about −189° C., about −190° C., about −191° C., about −192° C., about −193° C., about −194° C., about −195° C., about −196° C., about −197° C., about −198° C., and about −199° C. Still further, the temperature can be in any range derived from any two of the above stated values. For example, the at least a portion of the separated syngas exiting the second heat exchange unit can have a temperature in the range from about 155° C. to about −190° C., or from about −168° C. to about −172° C., or from about −170° C. to about −195° C.

In some aspects, the at least a portion of the separated syngas exiting the second heat exchange unit can comprise at least a first portion of the separated syngas and at least a second portion of the separated syngas.

In some aspects, the second heat exchange unit is in communication with the first heat exchange unit to allow energy recycling within the process. In certain aspects, the second heat exchange unit is in thermal communication with the first heat exchange unit. In one aspect, the at least a first portion of the separated syngas is further recycled back to a Fischer-Tropsch reactor (104). In certain aspects, the recycling comprises passing the at least a first portion of the separated syngas utilizing line (120) through the first heat exchange unit (108). In some aspects, the first heat exchange device of the first heat exchange unit that the at least a first portion of the separated syngas enters is the last heat exchange device of the first heat exchange unit that the first product stream passes through. In certain aspects, a flow of the first product stream in the first heat exchange unit is counter-flow to a flow of the at least a first portion of the separated syngas. In certain aspects, the passing of the at least a first portion of the separated syngas through the first heat exchange unit transfers a heat released by the first product stream to the at least a first portion of the separated syngas. It is understood that the passing through the first heat exchange unit, a temperature of the at least a first portion of the separated syngas rises due to the heat exchange with the first product stream. In some aspects, the at least a first portion of the separated syngas collected in the syngas recovery unit (122), after the exit from the first heat exchange unit has a temperature in the range from about 10° C. to about 50° C., including exemplary values of about 15° C., about 20° C., about 25° C., about 30° C., about 35° C., about 40° C., and about 45° C. Still further, the temperature can be in any range derived from any two of the above stated values. For example, the temperature of the at least a first portion of the separated syngas collected on the exit from the first heat exchange unit can be about 15° C. to about 40° C., or from about 20° C. to about 50° C.

In certain aspects, the at least a second portion of the separated syngas is transferred by line (121) to the methane wash unit (150). According to the aspects disclosed herein, the method further comprises separating at least a portion of hydrogen (H₂) from the at least a second portion of the separated syngas in the methane wash unit. In some other aspects, the methane wash unit (150) further utilizes at least a second portion of the methane exiting the demethanizer (130). The methane wash units are known in the art. Methane wash units, for example, described in U.S. Pat. Nos. 4,102,659 and 6,578,377, which are incorporated herein by their entirety, specifically for their disclosure regarding methane wash units. The methane wash unit (150) can comprise a cryogenic column separating methane and carbon monoxide from hydrogen. In some aspects, the at least a second portion of the separated syngas is contacted in a counter-flow with a methane wash liquid in a first adsorption zone of the methane wash unit to recover at least a portion of hydrogen gas as overhead and bottoms liquid comprising methane, carbon monoxide and residual hydrogen. In other aspects, the bottoms liquid from the first absorption zone is throttled to a lower pressure and is contacted in a counter flow with a hydrogen rich vapor in a second absorption zone for absorption of carbon monoxide from the hydrogen rich vapor by the throttled bottoms liquid therein to recover residual hydrogen gas as overhead and bottoms liquid enriched in carbon monoxide. In other aspects the bottoms liquid recovered from the second absorption zone are fractionated in a fractionation zone to recover overhead gas comprising carbon monoxide and bottoms liquid comprising methane. In yet other aspects, at least a portion of the bottoms liquid recovered from the fractionation zone can be recycled to the first absorption zone as the methane wash liquid. In yet further aspects, the recovered hydrogen is collected in a hydrogen recovery unit (152). In still further aspects, a mixture (154) of the removed methane and carbon monoxide can be further recycled into the cryogenic separation unit (114).

In yet another aspect, the at least a second portion of the separated syngas can be returned back to the cryogenic separation unit (114) to provide auxiliary reflux.

According to the aspects disclosed herein, the second product stream comprising methane and C2-C4 hydrocarbons separated from the syngas passes through a third heat exchange unit (124) to reach a temperature in the range from about −70° C. to about −150° C., including exemplary values of about −75° C., about −80° C., about −85° C., about −90° C., about −95° C., about −100° C., about −105° C., about −110° C., about −115° C., about −120° C., about −125° C., about −130° C., about −135° C., about −140° C., and about −145° C. Still further, the temperature can be in any range derived from any two of the above stated values. For example, the temperature of the second product stream collected at the exit (126) from the third heat exchange unit can be about −75° C. to about −85° C., or from about −80° C. to about −95° C., or from about −100° C. to about −110° C. In certain aspects, the third heat exchange unit (124) is in communication with the first heat exchange unit (108). In other aspects, the third heat exchange unit is in thermal communication with the first heat exchange unit. It is further understood that the third heat exchange unit (124) can be in communication with one or more heat exchange devices (128) present in the first heat exchange unit (108). It is further understood that the third heat exchange unit can comprise one or more of a parallel-flow heat exchange device, a counter-flow heat exchange device, a cross-flow heat exchange device, or any combination thereof. In some aspects, the second product stream exiting the third heat exchange unit can comprise at least a first portion of the second product stream and at least a second portion of the second product stream. In some aspects, the at least a first portion of the second product stream is returned to the bottom of the cryogenic separation unit (114). At least a portion of the second product stream is returned to the bottom of the cryogenic separation unit (114) to be reheated to separate residual syngas, which is used to drive the distillation function of C1-C4 hydrocarbon recovery (second product stream).

According to further aspects of this disclosure, the at least a second portion of the second product stream (126) enters a demethanizer (130). In some aspects, the at least a portion of the methane is separated from the at least a second portion of the second product stream in the demethanizer. Demethanizers are known in the art. As one of ordinary skill in the art would readily appreciate, the demethanizer allows separation of at least a portion of methane from higher hydrocarbons, for example and without limitation, from C2-C9 and C+10 hydrocarbons. The demethanizer can be a fractionation column, which use distillation separation technologies for methane separation from higher hydrocarbons. Demethanizers, for example, described in U.S. Pat. Nos. 4,270,940 and 5,953,935, which are incorporated herein by their entirety, specifically for their disclosure regarding demethanizers. According to the aspects of the disclosure, separating at least a portion of the methane from the at least a second portion of the second product stream in the demethanizer produces a third product stream (136, 146) comprising C2-C4 hydrocarbons. In some aspects, the at least a portion of the separated methane exiting the top part of the demethanizer can pass through a fourth heat exchange unit (132). It is further understood that the fourth heat exchange unit can comprise one or more of a parallel-flow heat exchange device, a counter-flow heat exchange device, a cross-flow heat exchange device, or any combination thereof. In certain aspects, the fourth heat exchange unit can be in communication with the first heat exchange unit. In other aspects, the fourth heat exchange unit can be in thermal communication with the first heat exchange unit. It is further understood that the fourth heat exchange unit can be in communication with one or more heat exchange devices present in the first heat exchange unit. In some aspects, the at least a portion of the separated methane exiting the fourth heat exchange unit (132) can comprise at least a first portion, at least a second portion, and at least a third portion of the separated methane. Further, according to the aspects of this disclosure, the at least a first portion of the separated methane flow (134) passes through the first heat exchange unit thereby transferring a heat released by the first product stream to the at least a first portion of the separated methane. The at least a first portion of the separated methane collected in a methane recovery unit (138) is further transferred to a syngas generation unit (102). It is understood that the recycling of the at least a first portion of the separated methane to the syngas generation unit (102) and the at least a first portion of the separated syngas to the Fisher-Tropsch reactor (104) allows a continued loop of producing desirable olefins while minimizing waste and increasing yield and efficiency.

In further aspects, the at least a second portion of the separated methane exiting the fourth heat exchange unit is transferred by line (151) to the methane wash unit (150). In further aspects, the method disclosed herein comprises utilizing the at least a second portion of the separated methane in the methane wash unit (150). In certain aspects, at least a portion of the methane in the mixture of methane and carbon monoxide (154) removed from the methane wash unit (150) comprises the at least a second portion of the separated methane. According to the aspects of this disclosure, the method comprises separating at least a portion of H₂ from the at least a second portion of the separated syngas and methane in the methane wash unit, wherein at least a portion of methane in the methane wash unit comprises the at least a second portion of the separated methane.

In some aspects, the at least a second portion of the separated methane exiting the fourth heat exchange unit can be returned to the demethanizer as a top reflux. In other aspects, the at least a third portion of the separated methane exiting the fourth heat exchange unit can be returned to the demethanizer as a top reflux.

In other aspects, the third product stream can comprise at least a first portion of the third product stream and at least a second portion of the third product stream. In certain aspects, the at least a first portion of the third product stream is recycled back to the bottom of the demethanizer. At least a portion of the third product stream (136, 146) is returned to the bottom of the demethanizer (130) to be reheated to separate residual methane, which is used to drive the distillation function and the C2-C7 hydrocarbon recovery (third product stream). In yet other aspects, the second portion of the third product stream is transferred for further separation of ethylene, ethane, propylene, or propane, or a combination thereof. In yet other aspects, the second portion of the third product stream is transferred for further separation of ethylene, ethane, propylene, or propane, or a combination thereof.

In certain aspects, the process described herein further comprises separating ethylene, ethane, propylene, or propane, or a combination thereof from the at least a second portion of the third product stream. In yet other aspects, the process described herein comprises separating ethylene, ethane, propylene, or propane, or a combination thereof from the third product stream. It is understood that any separation methods known in the art can be employed. For example and without limitation, demethanizers or depropanizers can be employed for the olefin separation.

In some aspects, the yield of C2-C4 hydrocarbon recovery is from about 80% to about 100%, including exemplary values of about 85%, about 90%, about 95%, about 98%, about 99%, about 99.5%, and about 99.9%.

### 4. Aspects

In view of the described catalyst and catalyst compositions and methods and variations thereof, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

Aspect 1: A method comprising the steps of: a) providing a first product stream comprising syngas, methane, and C2-C4 hydrocarbons, wherein the first product stream has a first temperature; b) lowering the first temperature of the first product stream to a second temperature in a first heat exchange unit; c) separating at least a portion of the syngas from the first product stream in a cryogenic separation unit, thereby producing a second product stream comprising methane and C2-C4 hydrocarbons; d) separating at least a portion of the methane in the second product stream in a demethanizer, thereby producing a third product stream comprising C2-C4 hydrocarbons; e) recycling the at least a portion of the separated syngas to a Fischer-Tropsch reactor; and f) recycling the at least a portion of the separated methane to a syngas generation unit.

Aspect 2: The method of aspect 1, further comprises the step of separating at least a portion of H₂ from the at least a portion of the separated syngas in a methane wash unit, thereby producing a methane wash product comprising methane and carbon monoxide, wherein at least a portion of separated methane in the method of claim 1 is used to wash the at least a portion of the separated syngas in the methane wash unit.

Aspect 3: The method of aspect 1, wherein the separated syngas has a third temperature, wherein the method further comprises the steps of: lowering the third temperature of at least a portion of the separated syngas to a fourth temperature via a N₂ refrigeration loop and a second heat exchange unit; and recycling energy from the portion of the separated syngas with the fourth temperature to the first product stream comprising syngas, methane, and C2-C4 hydrocarbons via the first heat exchange unit.

Aspect 4: The method of any one of aspects 1-3, wherein the first temperature is in the range from about 10° C. to about 50° C.

Aspect 5: The method of any one of aspects 1-4, wherein the first product stream has a first pressure in the range from about 20 bar to about 50 bar.

Aspect 6: The method of any one of aspects 1-5, wherein the second temperature is in the range from about −120° C. to about −170° C.

Aspect 7: The method of any one of aspects 1-6, wherein the first heat exchange unit is in communication with a first refrigeration system.

Aspect 8: The method of any one of aspects 1-7, wherein the first heat exchange unit comprises one or more heat exchange devices.

Aspect 9: The method of any one of aspects 1-8, wherein the first heat exchange unit comprises at least two heat exchange devices.

Aspect 10: The method of any one of aspects 1-9, wherein the lowering of the first temperature of the first product stream to the second temperature separates a hydrocarbon product comprising C2-C7 hydrocarbons from the first product stream, wherein the separated hydrocarbon product is in a liquid form.

Aspect 11: The method of aspect 10, wherein the method further comprises the step of introducing the liquid hydrocarbon product comprising C2-C7 hydrocarbons into the demethanizer.

Aspect 12: The method of any one of aspects 1, 2, or 4-11, wherein the method further comprises the step of passing the at least a portion of the separated syngas through a second heat exchange unit and wherein the temperature of the at least a portion of the separated syngas is in the range from about −150° C. to about −170° C.

Aspect 13: The method of any one of aspects 1, 2, or 4-12, wherein the method further comprises lowering the temperature of the at least a portion of the separated syngas to a temperature in the range of about −170° C. to about −200° C.

Aspect 14: The method of aspect 13, wherein the lowering of the temperature of the at least a portion of the separated syngas is performed in a gas expansion unit.

Aspect 15: The method of any one of aspects 12-14, wherein the method further comprises passing the at least a portion of the separated syngas having a temperature of −170° C. to about −200° C. and exiting the gas expansion unit through the second heat exchange unit, thereby increasing the temperature of the separated syngas to −150° C. to about −170° C.

Aspect 16: The method of any one of aspects 1-15, wherein the second heat exchange unit is in communication with the first heat exchange unit.

Aspect 17: The method of any one of aspects 1, 2, or 4-16, wherein the recycling of the at least a portion of the separated syngas to the Fisher-Tropsch reactor comprises passing the at least a portion of the separated syngas through the first heat exchange unit; thereby transferring a heat released by the first product stream to the at least a portion of the separated syngas; thereby also lowering the temperature of the first product stream.

Aspect 18: The method of any one of aspects 1, 2, or 4-17, wherein the at least a portion of the separated syngas is at a temperature in the range of about 10° C. to about 50° C. after passing through the first heat exchange unit.

Aspect 19: The method of any one of aspects 1-18, wherein a flow of the first product stream in the first heat exchange unit is a counter-flow to a flow of the at least a portion of the separated syngas being recycled to the Fisher-Tropsch reactor.

Aspect 20: The method of any one of aspects 1-19, wherein the second product stream comprising methane and C2-C4 hydrocarbons passes through a third heat exchange unit to reach a temperature in the range from about −70° C. to about −100° C.

Aspect 21: The method of any one of aspects 1-20, wherein the third heat exchange unit is in communication with the first heat exchange unit.

Aspect 22: The method of any one of aspects 1-21, wherein the method further comprises passing the at least a portion of the separated methane through a fourth heat exchange unit.

Aspect 23: The method of any one of aspects 1-22, wherein the fourth heat exchange unit is in communication with the first heat exchange unit.

Aspect 24: The method of any one of aspects 1-21, wherein the method further comprises passing the at least a portion of the separated methane through a fourth heat exchange unit.

Aspect 24: The method of any one of aspects 1-23, wherein the method further comprises passing the at least a portion of the separated methane through the first heat exchange unit thereby transferring a heat released by the first product stream to the at least a portion of the separated methane, thereby lowering the temperature of the first product stream.

Aspect 25: The method of any one of aspects 1-24, wherein the method further comprises separating ethylene, ethane, propylene, or propane, or a combination thereof from at least a portion of the third product stream.

Aspect 26: The method of any one of aspects 2-25, wherein the method further comprises utilizing at least a portion of the separated methane in the methane wash unit.

Aspect 27: The method of any one of aspects 2-26, wherein the method further comprises separating at least a portion of H₂ from at least a portion of the separated syngas in the methane wash unit, wherein at least a portion of methane in the methane wash unit comprises the at least a portion of the separated methane.

Aspect 28: The method of any one of aspects 2-27, wherein the method further comprises recycling of the methane wash product comprising methane and carbon monoxide to the cryogenic separation unit.

Aspect 29: The method of any one of aspects 3-11, 14, 15, or 18-25, wherein the third temperature is in the range of −155° C. to about −160° C.

Aspect 30: The method of any one of aspects 3-11, 14, 15, 18-25, or 29, wherein the fourth temperature is in the range of about −150° C. to about −200° C.

Aspect 31: The method of any one of aspects 3-11, 14, 15, 18-25, or 29-30, wherein recycling energy of the at least a portion of the separated syngas with the fourth temperature comprises passing the at least a portion of the separated syngas through the first heat exchange unit thereby transferring a heat released by the first product stream to the at least a portion of the separated syngas; thereby also lowering the temperature of the first product stream.

Aspect 32: The method of any one of aspects 3-11, 14, 15, 18-25, or 29-31, wherein after passing the at least a portion of the separated syngas through the first heat exchange unit, a temperature of the at least a portion of the separated syngas is in the range of about 10° C. to about 50° C.

Aspect 33: A system comprising: a) a syngas generation unit; b) a Fisher-Tropsch reactor; c) a first, a second, a third, and a fourth heat exchange unit, wherein at least one of the second, the third, and the fourth exchange units is in communication with the first heat exchange unit; d) a first refrigeration unit; wherein the first refrigeration unit is in communication with the first heat exchange unit; e) a cryogenic separation unit, wherein the cryogenic separation unit is in communication with the Fisher-Tropsch reactor; and f) a demethanizer, wherein the demethanizer is in communication with the syngas generation unit.

Aspect 34: The system of aspect 33, wherein the system further comprises: g) a methane wash unit, wherein the methane wash unit is in communication with the cryogenic separation unit and the demethanizer unit; h) a syngas recovery unit, wherein the syngas recovery unit is in communication with the Fisher-Tropsch reactor; and i) a methane recovery unit, wherein the methane recovery unit is in communication with the syngas generation unit.

Aspect 35: The system of aspect 33, wherein the system further comprises: g1) a N₂ refrigeration loop, wherein the N₂ refrigeration loop is in communication with the second heat exchange unit; h1) a syngas recovery unit, wherein the syngas recovery unit is in communication with the Fisher-Tropsch reactor; and i1) a methane recovery unit, wherein the methane recovery unit is in communication with the syngas generation unit.

Aspect 36: The system of any of aspects 33-35, wherein the syngas generation unit is in communication with the Fisher-Tropsch reactor.

Aspect 37: The system of any of aspects 33-35, wherein the cryogenic separation unit is in communication with the demethanizer.

1. A method comprising the steps of:
a) providing a first product stream comprising syngas, methane, and C2-C4 hydrocarbons, wherein the first product stream has a first temperature;
b) lowering the first temperature of the first product stream to a second temperature in a first heat exchange unit;
c) separating at least a portion of the syngas from the first product stream in a cryogenic separation unit, thereby producing a second product stream comprising methane and C2-C4 hydrocarbons;
d) separating at least a portion of the methane in the second product stream in a demethanizer, thereby producing a third product stream comprising C2-C4 hydrocarbons;
e) recycling the at least a portion of the separated syngas to a Fischer-Tropsch reactor; and
f) recycling the at least a portion of the separated methane to a syngas generation unit.

2. The method of claim 1, further comprises the step of separating at least a portion of H₂ from the at least a portion of the separated syngas in a methane wash unit, thereby producing a methane wash product comprising methane and carbon monoxide, wherein at least a portion of separated methane in the method of claim 1 is used to wash the at least a portion of the separated syngas in the methane wash unit.

3. The method of claim 1, wherein the separated syngas has a third temperature,
wherein the method further comprises the steps of:
lowering the third temperature of at least a portion of the separated syngas to a fourth temperature via a N₂ refrigeration loop and a second heat exchange unit; and
recycling energy from the portion of the separated syngas with the fourth temperature to the first product stream comprising syngas, methane, and C2-C4 hydrocarbons via the first heat exchange unit.

4. The method of claim 1, wherein the first heat exchange unit is in communication with a first refrigeration system.

5. The method of claim 1, wherein the lowering of the first temperature of the first product stream to the second temperature separates a hydrocarbon product comprising C2-C7 hydrocarbons from the first product stream, wherein the separated hydrocarbon product is in a liquid form.

6. The method of claim 1, wherein the method further comprises the step of introducing the liquid hydrocarbon product comprising C2-C7 hydrocarbons into the demethanizer.

7. The method of claim 1, wherein the method further comprises the step of passing the at least a portion of the separated syngas through a second heat exchange unit and wherein the temperature of the at least a portion of the separated syngas is in the range from about −150° C. to about −170° C.

8. (canceled)

9. (canceled)

10. The method of claim 7, wherein method further comprises passing the at least a portion of the separated syngas having a temperature of −170° C. to about −200° C. and exiting the gas expansion unit through the second heat exchange unit, thereby increasing the temperature of the separated syngas to −150° C. to about −170° C.

11. The method of claim 1, wherein the recycling of the at least a portion of the separated syngas to the Fisher-Tropsch reactor comprises passing the at least a portion of the separated syngas through the first heat exchange unit; thereby transferring a heat released by the first product stream to the at least a portion of the separated syngas; thereby also lowering the temperature of the first product stream.

12. The method of claim 1, wherein a flow of the first product stream in the first heat exchange unit is a counter-flow to a flow of the at least a portion of the separated syngas being recycled to the Fisher-Tropsch reactor.

13. The method of claim 1, wherein the second product stream comprising methane and C2-C4 hydrocarbons passes through a third heat exchange unit to reach a temperature in the range from about −70° C. to about −100° C.

14. The method of claim 1, wherein the third heat exchange unit is in communication with the first heat exchange unit.

15. (canceled)

16. The method of claim 1, wherein the method further comprises passing the at least a portion of the separated methane through the first heat exchange unit thereby transferring a heat released by the first product stream to the at least a portion of the separated methane, thereby lowering the temperature of the first product stream.

17. (canceled)

18. The method of claim 2, wherein the method further comprises utilizing at least a portion of the separated methane in the methane wash unit.

19. The method of claim 2, wherein the method further comprises separating at least a portion of H₂ from at least a portion of the separated syngas in the methane wash unit, wherein at least a portion of methane in the methane wash unit comprises the at least a portion of the separated methane.

20. The method of claim 2, wherein the method further comprises recycling of the methane wash product comprising methane and carbon monoxide to the cryogenic separation unit.

21. The method of claim 3, wherein recycling energy of the at least a portion of the separated syngas with the fourth temperature comprises passing the at least a portion of the separated syngas through the first heat exchange unit thereby transferring a heat released by the first product stream to the at least a portion of the separated syngas; thereby also lowering the temperature of the first product stream.

22. (canceled)

23. A system comprising:
a) a syngas generation unit;
b) a Fisher-Tropsch reactor;
c) a first, a second, a third, and a fourth heat exchange unit, wherein at least one of the second, the third, and the fourth exchange units is in communication with the first heat exchange unit;
d) a first refrigeration unit; wherein the first refrigeration unit is in communication with the first heat exchange unit;
e) a cryogenic separation unit, wherein the cryogenic separation unit is in communication with the Fisher-Tropsch reactor; and
f) a demethanizer, wherein the demethanizer is in communication with the syngas generation unit.

24. The system of claim 23, wherein the system further comprises:
g) a methane wash unit, wherein the methane wash unit is in communication with the cryogenic separation unit and the demethanizer unit;
h) a syngas recovery unit, wherein the syngas recovery unit is in communication with the Fisher-Tropsch reactor; and
i) a methane recovery unit, wherein the methane recovery unit is in communication with the syngas generation unit.

25. The system of claim 23, wherein the system further comprises:
g1) a N₂ refrigeration loop, wherein the N₂ refrigeration loop is in communication with the second heat exchange unit;
h1) a syngas recovery unit, wherein the syngas recovery unit is in communication with the Fisher-Tropsch reactor; and
i1) a methane recovery unit, wherein the methane recovery unit is in communication with the syngas generation unit.

26. (canceled)

27. (canceled)
